(19) United States
(12) Patent Application Publication
(10) Pub. No.: US 2026/0096791 A1
(43) Pub. Date: Apr. 9, 2026

(54) PORTABLE X-RAY SYSTEM

(71) Applicants: Timothy L. Moulton, Newport, RI (US); Sung Park, Brookline, MA (US)

(72) Inventors: Timothy L. Moulton, Newport, RI (US); Sung Park, Brookline, MA (US)

(21) Appl. No.: 19/345,741

(22) Filed: Sep. 30, 2025

Related U.S. Application Data
(63) Continuation of application No. PCT/US25/46084, filed on Sep. 12, 2025.
(60) Provisional application No. 63/695,634, filed on Sep. 17, 2024.

(51) Int. Cl. A61B 6/00; A61B 6/02; A61B 6/06; A61B 6/46

(52) U.S. Cl. CPC A61B 6/4405; A61B 6/025; A61B 6/06; A61B 6/4452; A61B 6/462; A61B 6/547; A61B 6/56

(57) ABSTRACT

A system for x-ray imaging includes a source module comprising an x-ray source, a high voltage power source for running the x-ray source for pulse-based operation, a system of x-ray opaque leaves that can be moved independently to provide an aperture for x-rays produced by the x-ray source, an optical camera system capable of accurately determining the orientation and distance of a device of know size with a known layout of markers, a detector module separate from the source module and comprising an electronic x-ray detector that produces x-ray image data from x-rays produced by the x-ray source, a computing system capable of processing the x-ray image data from the x-ray detector to produce images, a computer screen capable of displaying the images produced by the portable computer system with high dynamic range, a communication connection between at least the source module and the detector module.

### CROSS-REFERENCE TO RELATED APPLICATION(S)

This patent application is a continuation of International Patent Application No. PCT/US25/46084 entitled PORTABLE X-RAY SYSTEM filed Sep. 12, 2025, which claims the benefit of U.S. Provisional Ser. No. 63/695,634 entitled PORTABLE X-RAY SYSTEM filed Sep. 17, 2024, each of which is hereby incorporated herein by reference in its entirety.

### FIELD OF THE INVENTION

The invention generally relates to a portable x-ray system.

### BACKGROUND OF THE INVENTION

X-ray machines are generally stationary devices (e.g., in hospitals or other healthcare facilities). Portable x-ray systems do exist, but they are generally complex, expensive, and require expert operators.

### SUMMARY OF VARIOUS EMBODIMENTS

In accordance with one embodiment of the invention, a system for x-ray imaging includes a source module comprising an x-ray source, a high voltage power source for running the x-ray source for pulse-based operation, a system of x-ray opaque leaves that can be moved independently to provide an aperture for x-rays produced by the x-ray source, an optical camera system capable of accurately determining the orientation and distance of a device of know size with a known layout of markers, a detector module separate from the source module and comprising an electronic x-ray detector that produces x-ray image data from x-rays produced by the x-ray source, a computing system capable of processing the x-ray image data from the x-ray detector to produce images, the computing system providing a user interface (UI), a computer screen capable of displaying the images produced by the portable computer system with high dynamic range, and a communication connection between at least the source module and the detector module.

In various alternative embodiments, there may be no mechanical connection between the x-ray source and the electronic x-ray detector. The communication connection between the source and detector modules may be wireless. The markers may be on extendable or moveable arms to improve visibility, wherein the movement of the marker extensions are instrumented to provide accurate positioning information that is conveyed to the optical tracking system. The x-ray source and/or the x-ray detector may be temporarily, semi-permanently, or permanently mounted on an existing structure or general use support structure.

The images may be diagnostic images such as of minor extremities such as the hands and feet, the torso or other major body parts, the head or other major extremities, diagnostic images made in various poses including weight bearing poses, diagnostic images having variable magnification based on the relative positions of the x-ray source and the x-ray detector, or diagnostic images for veterinary purposes. A diagnostic image maybe be exchanged or uploaded to a medical record repository or another device for review, e.g., via a wired network that connects to the source or the detector module, via a wireless network that is different than the network that connects to the source or the detector module, or via a wireless network that is the same as the network that connects to the source or the detector module. Diagnostic images may be directly integrated into a central repository that can retrieve or restore medical records from such a repository, e.g., through a hardwired network link, through a wireless network interface, or through the same wired or wireless interface that link the source and detector units.

The images may be tomosynthesis based 3D images from a series of individual images and the corresponding position and orientation information. The resultant image may be displayed on the computer screen in a similar manner to a conventional digital camera. Multiple x-ray images may be aligned and used in a tomosynthesis reconstruction by way of an array of radio-detectable fiducials that are mounted to the anatomy of the subject. Multiple x-ray images may be aligned and used in a tomosynthesis reconstruction by way of an array of optical fiducials that are mounted to the anatomy of the subject and aligned using the same optical tracking system used to align the detector to the x-ray source. Multiple x-ray exposures may be stitched together into one larger diagnostic image by use of the optical tracking system and known displacement of a grid x-ray detector. Multiple x-ray exposures may be produced where both the detector and source are moved for each exposure but located and stitched together by the use of the optical tracking system determining the position of the source and the detector with known markers and the use of a second set of visible markers that are stationary with respect to the patient between captures, but remain in the optical field of view. An x-ray image may be created with a predetermined source and detector distance and orientation through the use of user cues to orient and position the source module with respect to the detector module. A diagnostic image equivalent to a linear scan image may be created throughout the use of user cues, prescribed camera motion, and a continually adaptive slit configuration of the x-ray opaque leaves. For example, the x-ray opaque leaves may be continually and near-instantaneously adjusted using the information from the optical tracking system and a known relationship to the focal spot of the x-ray source to ensure only x-rays that are aimed for the detector are permitted to emanate from the source.

The system may only initiate the x-ray source if the x-ray opaque leaves are in the proper orientation. Initiation of the x-ray source may be prevented if the optical tracking system is not able to obtain adequate tracking information. Initiation of the x-ray source may be prevented if the x-ray source is determined to be too close to the detector.

An optical image may be taken concurrently with the diagnostic x-ray image and stored as a single record including the orientation and distance information. The x-ray images may be combined with optical views to create a tomosynthesis reconstruction with a texture mapped surface from the optical images. Texture mapped tomosynthesis reconstruction may be used to align future additional images and data sets with visual markers on the patient's anatomy. Multiple source and detector units may be combined to align and combine x-ray exposures and optical images for additional information. The system may use multiple source or detector units to increase the field of view, to aid in tomosynthesis, or to locally increase magnification around an anatomical area of interest. The user interface may prompt the user to alter specific conditions to improve or augment imaging performance. For example, the UI may be configured to determine the relative position between the source and the detector modules and prompt the user to position the source module for orthogonal imaging. The UI may be configured to determine the relative position between the source and the detector modules and prompt the user to position the source module for additional projections to increase the effective field of view. The UI may be configured to determine the relative position between the source and the detector modules and prompt the user to position the source module for additional projections in aid of building a data set for tomosynthesis. The UI may be configured to determine the relative position between the source and the detector modules and prompt the user to position the source module for ideal magnification. The UI may be configured to determine the relative position between the source and the detector modules and display the effective magnification. The UI may be configured to determine the relative position between the source and the detector modules and prompt the user to position the source module to comply with predetermined safety or functional parameters. The UI may be configured to determine the relative position between the source and the detector modules and prompt the user to position the source module to align with a previous imaging exercise. The UI may be configured to determine the relative position between the source and the detector modules and prompt the user to position the source module to align with a previous imaging exercise based on a combination of the source and detector position and optical landmarks, either anatomically based or artificial markers. The user interface (UI) may be used to alter specific conditions to improve or augment imaging performance, alter doss or other change other beneficial conditions, e.g., using the touchscreen to set the energy or beam current of the source, using markers manipulated on the screen to collimate the x-ray beam to an area smaller than the full detector size, or using markers manipulated on the screen to collimate the x-ray beam to an area smaller than the full detector size and indicate the effective area in real time on the screen, in some instances as an overlay on the optical image. The UI may be separable from both the source and detector module. The UI may be either temporarily or permanently fixed to the source module. The UI may be on another piece of hardware, such as a tablet or smartphone.

The system may incorporate additional sensing technologies for alignment and imaging, e.g., a lidar sensor, stereoscopic cameras, infrared or thermal camera, or an ultrasonic distance measurement device. Alignment and imaging information may be overlaid on the x-ray diagnostic image. Additional sensor information may be used to assist in a tomographic or tomosynthetic reconstruction.

The x-ray source may include a carbon nanotube based x-ray source. The x-ray source may not require external cooling. The x-ray source may be be rapidly turned on and off. The system of x-ray opaque leaves may be motor actuated. The system of x-ray opaque leaves may include confirmatory position sensing. The electronic x-ray detector may include a solid-state flat panel detector, and the electronic x-ray detector may have a wide dynamic range, a small pixel size, and a large active area.

Both the source module and the detector module may be handheld modules. The power source may be part of the source module or may be separate from the source module. The system of x-ray opaque leaves may be part of the source module. The optical camera system may be part of the source module. The computer screen may be part of the source module. The aperture may include a quadrilateral aperture.

In accordance with another embodiment of the invention, an x-ray source module for a portable x-ray system includes a computing system, an x-ray source, a system of x-ray opaque leaves that can be moved independently to provide an aperture for x-rays produced by the x-ray source, and a communication interface for communication with a separate x-ray detector.

In accordance with another embodiment of the invention, an x-ray source module for a portable x-ray system includes a housing configured to attach to a separate portable computer, an x-ray source attached to the housing, a system of x-ray opaque leaves that can be moved independently to provide an aperture for x-rays produced by the x-ray source, and a communication interface for communication with the portable computer. The x-ray source module may be configured to receive at least some electrical power from the portable computer.

Additional embodiments may be disclosed and claimed.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 is a schematic diagram of a portable x-ray system in accordance with various embodiments.

FIG. 2 is a schematic diagram showing posable leaves 114 in accordance with certain embodiments.

FIG. 3 is a schematic diagram showing an example of how posable leaves can be used to produce a field of view based on the posable optical markers of the detector subsystem.

FIG. 4 is a schematic diagram showing general use of posing the detector subsystem behind a subject to be inspected with the optical markers visible to the sides of the area to be examined while pointing the handheld subsystem at the detector subsystem.

FIG. 5 is a schematic diagram showing the portable computer/housing including an interface for a tripod.

FIG. 6 is a schematic diagram depicting three handheld subsystems being used in conjunction with one or more detector subsystems, e.g., to capture a larger x-ray image and/or images from different angles.

FIG. 7 is a schematic block diagram illustrating at least one embodiment of a computing system configured as a portable (i.e., mobile) device consistent with the present disclosure.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

Certain embodiments provide a portable x-ray system. Generally speaking, components of the system include at least one source of x-ray photons, a system of poseable x-ray opaque leaves (which, for convenience, may be referred to herein as “collimator” leaves), at least one detached grid of x-ray detectors, an optical tracking system, and a portable computing system. It should be noted that the optical tracking system could be an integral component of the portable computing system, e.g., an in-built camera of the portable computing system.

In certain embodiments, these system components are split into two main subsystems, specifically (1) a handheld subsystem that includes one or more x-ray photon sources, the computing system, the optical tracking system, and the poseable leaves as well as communication system(s) (e.g., wireless communication capabilities such as Bluetooth, WiFi, NFC, etc.), housing, and user interface, and (2) a separate detector subsystem that includes one or more x-ray detector grids and preferably also various posable optical markers as well as communication system(s) (e.g., wireless communication systems) and one or more mounts or mounting interfaces (e.g., for a tripod or other mounting system).

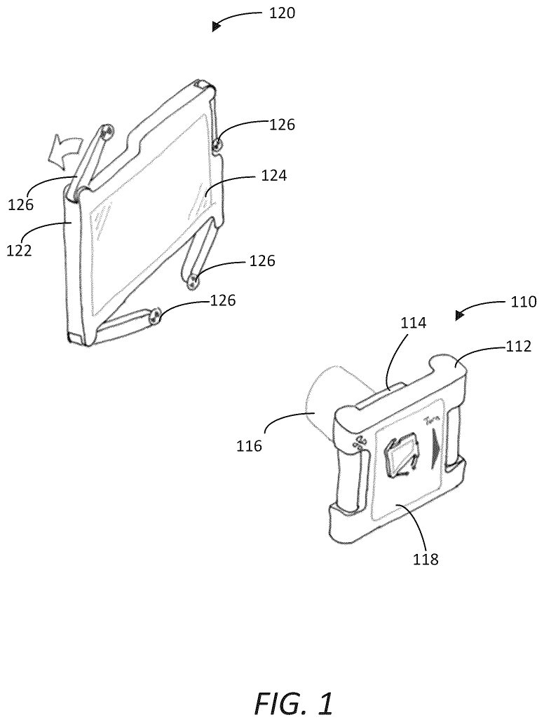

FIG. 1 is a schematic diagram of a portable x-ray system in accordance with various embodiments. Among other things, the handheld subsystem 110 includes a portable computer/housing 112, posable leaves 114, x-ray photon source 116, and display 118 (which may be part of the user interface, e.g., a touchscreen display allowing for both input and output). It should be noted that the posable leaves 114 and/or the x-ray photon source 116 may be integral with the portable computer/housing 112 or may be separate components that may be attached to the portable computer/housing 112 (e.g., a case or other separate components that attaches onto or otherwise can be used with a portable computer such as a tablet computer or smartphone, which may be powered by the portable computer or have its own power supply, and which may communicate with the portable computer via wired or wireless communication) or in some cases possibly used separately from the portable computer/housing 112. Among other things, the detector subsystem 120 includes a housing 122 supporting x-ray detector grid 124 and a plurality of posable optical markers 126.

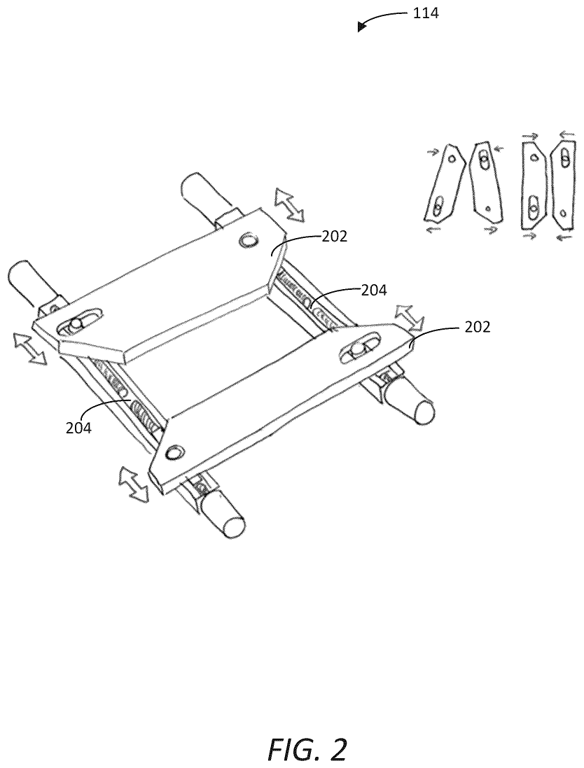

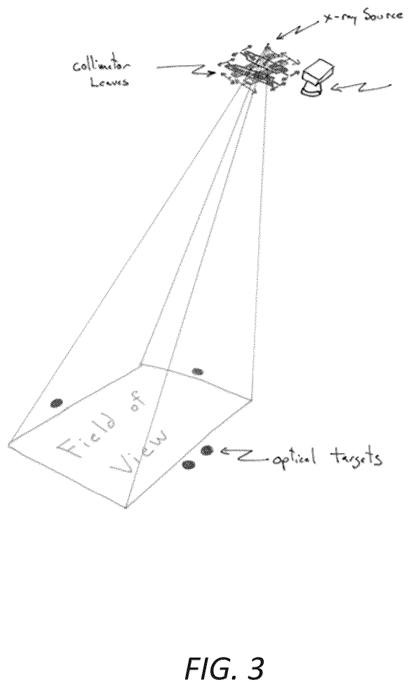

FIG. 2 is a schematic diagram showing posable leaves 114 in accordance with certain embodiments. Here, the posable leaves 114 include two opaque panels 202 each associated with two actuators 204 that can be operated by the portable computer 112 to move the opaque panels 202 into various positions (e.g., see the inset in FIG. 2 for an example of how the opaque panels 202 may be moved to create different apertures as well as FIG. 3 for an example of how the posable leaves 114 can be used to produce a field of view based on the posable optical markers 126 of the detector subsystem 120, e.g., with the field of view produced automatically by the portable computer 110 based on optical images of the posable optical markers 126).

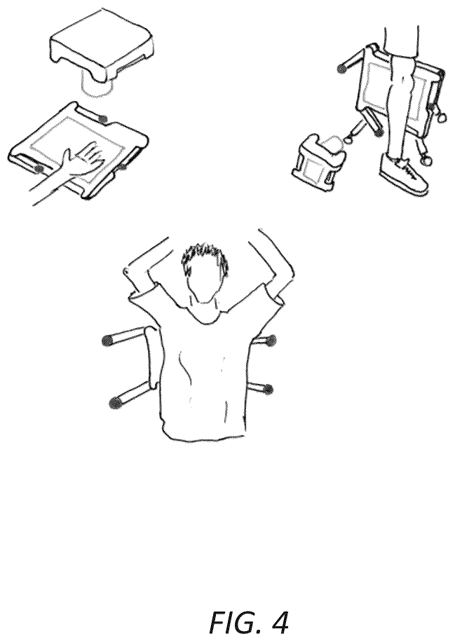

As shown schematically in FIG. 4, the system is generally used by posing the detector subsystem 120 behind a subject to be inspected with the optical markers visible to the sides of the area to be examined while pointing the handheld subsystem at the detector subsystem. FIG. 4 shows examples for x-ray of a hand, a leg, and a torso. While in use and using the markers on the detector subsystem 120, the portable computer and optical camera in the handheld subsystem 110 track the detector subsystem 120 with respect to the x-ray photon source 116, compute the angles and distance to the target detector 124, and position the poseable x-ray opaque leaves 114 so that emitted x-rays that are not destined for the detector 124 are absorbed. When an image is to be taken, the portable subsystem 110 confirms that the x-ray opaque leaves 114 are in the proper position, readies the detector subsystem 120 through the communication (e.g., wireless) connection, records the relative position of the handheld subsystem 110 and the detector subsystem 120, triggers the x-ray photon source 116, downloads the data from the detector subsystem 120 through the communication (e.g., wireless) connection, and displays the image on display 118. Additionally or alternatively, the image could be transmitted to a separate display and/or to a remote site for analysis or storage.

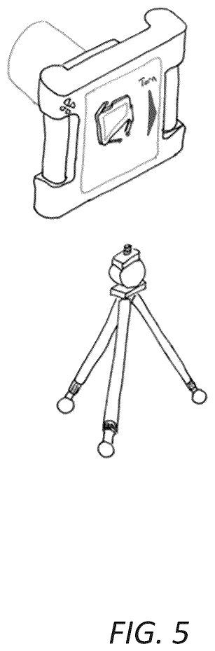

As discussed above, the portable computer/housing 102 may include any of various mounts or mounting interfaces. As but one example, FIG. 5 is a schematic diagram showing the portable computer/housing 102 including an interface for a tripod, although it should be noted that the portable computer/housing 102 could include one or more integral mounts (e.g., an attached tripod, stand, etc.) and/or other types of mounting interfaces (e.g., for mounting on a wall or other surface).

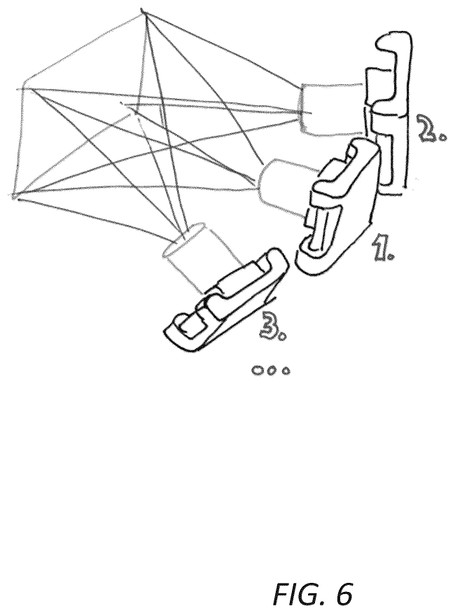

It should be noted that embodiments may allow for multiple handheld subsystems 110 and/or multiple detector subsystems 120 to be used in conjunction with one another. For example, FIG. 6 depicts three handheld subsystems 110 being used in conjunction with one or more detector subsystems 120, e.g., to capture a larger x-ray image and/or images from different angles. Multiple images can be “stitched” together, e.g., to form one larger image and/or to produce an image with additional information or detail (e.g., a 3D image). For example, an image could be produced of the full length of the spine or full body image, or two orthogonal views could be captured, e.g., for breast or prostate.

Thus, without limitation, embodiments can include a system for x-ray imaging that has a source of x-ray energy, a grid of x-ray detectors, independent means of ensuring x-ray safety, a means of displaying an image, and a wireless connection. For example, the device can include an x-ray source that can be rapidly turned on and off (e.g., preferably a carbon nanotube based x-ray source or other source that does not require external cooling), a high voltage power source for running the x-ray source for pulse based operation (e.g., preferably one that can be portable, battery driven and fit in a handheld form factor), a system of x-ray opaque leaves that can be moved independently to provide an arbitrary quadrilateral aperture (e.g., preferably actuated by motors with confirmatory position sensing), an optical camera system capable of accurately determining the orientation and distance of a device of know size with a known marker layout, an electronic x-ray detector (e.g., preferably a solid-state flat panel detector with a wide dynamic range, a small pixel size and a large active area), a computer screen (e.g., preferably one capable of displaying diagnostic images with high dynamic range), and a wireless connection between the detector and the handheld source unit.

Preferably, there is no mechanical connection between the source and detector assemblies such that the two subsystems can be manipulated independently, although alternative embodiments could include a mechanical connection or at least be adapted for a mechanical connection to be added if needed or desired. The data connection between the source and detector assemblies can be either wired or wireless.

The system can be used to produce any type of diagnostic images, e.g., of minor extremities such as the hands and feet, of the torso or other major body parts, of the head or other major extremities, etc. The diagnostic images can be made in various poses, including weight bearing poses. The diagnostic images can have variable magnification, e.g., based on the position of the handheld subsystem. The diagnostic images may be for any type of purpose, e.g., human purposes, veterinary purposes, x-ray of inanimate objects such as packages, etc. The computer system may be capable of generating tomosynthesis based 3D images from a series of individual images and the corresponding position and orientation information. The detector panel markers may be on extendable or moveable arms to improve visibility, e.g., wherein the movement of the marker extensions can be instrumented to provide accurate positioning information that is conveyed to the optical tracking system.

The x-ray image may be displayed on a screen on the handheld device in a similar manner to a conventional digital camera image, e.g., such that the experience of using the handheld subsystem is similar to the experience of using a tablet or smartphone camera (e.g., taking an x-ray is just like taking a typical photo). The source module and/or the detector module may be temporarily, semi-permanently, or permanently mounted on an existing structure or general use support structure. Multiple images may be aligned and used in a tomosynthesis reconstruction by way of an array of radio-detectable fiducials that are mounted to the anatomy of the subject. Multiple images may be aligned and used in a tomosynthesis reconstruction by way of an array of optical fiducials that are mounted to the anatomy of the subject and aligned using the same optical tracking system used to align the detector to the x-ray source. Multiple x-ray exposures may be stitched together into one larger diagnostic image by use of the optical tracking system and known displacement of a grid x-ray detector. The system may allow multiple x-ray exposures where both the detector and source are moved for each exposure but located and stitched together by the use of the optical tracking system determining the position of the source and the detector with known markers and the use of a second set of visible markers that are stationary with respect to the patient between captures but remain in the optical field of view. The system may be used to create an x-ray image with a predetermined source and detector distance and orientation through the use of user cues to properly orient and position the source with respect to the detector. A diagnostic image equivalent to a linear scan image may be created through the use of user cues, prescribed camera motion, and a continually adaptive slit configuration of the x-ray opaque leaves. The x-ray opaque leaves may be continually and near-instantaneously adjusted using the information from the optical tracking system and a known relationship to the focal spot of the x-ray source to ensure only x-rays that are aimed for the detector are permitted to emanate from the source.

The system may include various protective features, e.g., to prevent activation of the x-ray source unless and until the system determines that it is safe to do so. Thus, for example, the system may prevent initiation of the x-ray source if the x-ray opaque leaves are not in the proper orientation, if the optical tracking system is not able to obtain adequate tracking information, if the x-ray source is determined to be too close to the detector, etc.

A diagnostic image may be exchanged or uploaded to a medical record repository or another device for review and/or storage, e.g., uploaded via a wired network that connects to the source or the detector module, uploaded via a wireless network that is different than the network that connects to the source or the detector module, or uploaded via a wireless network that is the same as the network that connects to the source or the detector module.

An optical image may be taken concurrent with the diagnostic x-ray image and stored, e.g., as a single record with the orientation and distance information. One or more x-ray images may be combined with one or more optical images such as to create a tomosynthesis reconstruction with a texture mapped surface from the optical images. Texture mapped tomosynthesis reconstruction may be used to align future additional images and data sets with visual markers on the patient's anatomy.

The system may directly integrate diagnostic images into a central repository, e.g., to retrieve or restore medical records from such a repository. This can be done, for example, through a hardwired network link, through a wireless network interface, or through the same wired or wireless interface that link the source and detector units.

The system may combine x-ray exposures and optical images from multiple sources and/or detector units for additional information, e.g., to increase the field of view, to aid in tomosynthesis, to locally increase magnification around an anatomical area of interest, etc.

The user interface (UI) may prompt the user to alter specific conditions to improve or augment imaging performance. For example, the device and UI may determine the relative position between the source and the detector modules and prompts the user to position the source module for orthogonal imaging. The device and UI may determine the relative position between the source and the detector modules and prompts the user to position the source module for additional projections to increase the effective field of view. The device and UI may determine the relative position between the source and the detector modules and prompts the user to position the source module for additional projections in aid of building a data set for tomosynthesis. The device and UI may determine the relative position between the source and the detector modules and prompts the user to position the source module for ideal magnification. The device and UI may determine the relative position between the source and the detector modules and displays the effective magnification. The device and UI may determine the relative position between the source and the detector modules and prompts the user to position the source module to comply with any required safety or functional parameters. The device and UI may determine the relative position between the source and the detector modules and prompts the user to position the source module to align with a previous imaging exercise. The device and UI may determine the relative position between the source and the detector modules and prompts the user to position the source module to align with a previous imaging exercise based on a combination of the source and detector position and optical landmarks, either anatomically based or artificial markers. The user interface (UI) may be used to alter specific conditions to improve or augment imaging performance, alter doss or other change other beneficial conditions. For example, the touchscreen may be used to set the energy or beam current of the source, markers manipulated on the screen may be used to collimate the x-ray beam to an area smaller than the full detector size, markers manipulated on the screen may be used to collimate the x-ray beam to an area smaller than the full detector size and indicates the effective area in real time on the screen in some instances as an overlay on the optical image, etc.

The system also may react to user inputs, e.g., the system may automatically reconfigure the aperture as the user moves the handheld subsystem, e.g., based on the view being captured by the camera, or as the user manipulates the image on the screen, e.g., zooming in, zooming out, drawing a boundary such as a box or circle around a portion to be x-rayed, etc. The user interface could provide instruction to the user, e.g., where to place the handheld subsystem and/or the detector subsystem, how to get more or less magnification, etc.

Embodiments may incorporate additional sensing technologies such as for alignment and imaging, e.g., a lidar sensor, stereoscopic cameras, an infrared or thermal camera, an ultrasonic distance measurement device, etc. The additional information may be overlaid on the x-ray diagnostic image. The additional sensor information may be used to assist in a tomographic or tomosynthetic reconstruction.

The UI may be separable from both the source and detector module, e.g., the UI may be either temporarily or permanently fixed to the source module, or the UI may be on another piece of hardware, such as a tablet or smartphone.

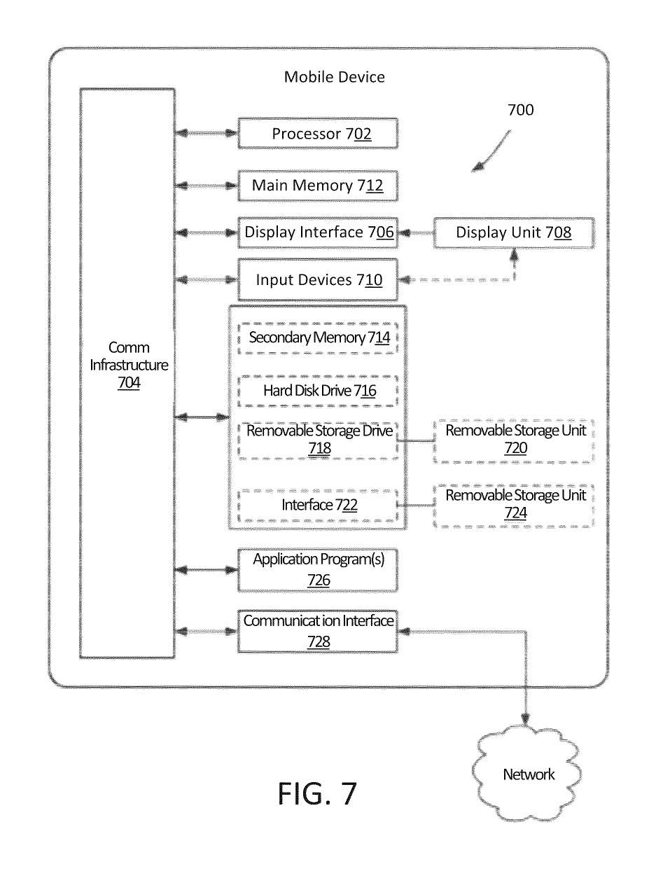

FIG. 7 is a schematic block diagram illustrating at least one embodiment of a computing system configured as a mobile (i.e., portable) device consistent with the present disclosure. The mobile device generally includes a computing system 700. As shown, the computing system 700 may include one or more processors 702, which can include, for example, at least one main processor (e.g., microprocessor, microcontroller, central processing unit, etc.) and optionally also at least one graphics processing unit (GPU) for performing any of various graphics-specific operations including, for example, producing tessellated graphics for display on display unit 708 (although in some embodiments the main processor may include a GPU and/or an extended instruction set of graphics instructions, or otherwise may perform graphics-specific operations such as in software). For convenience, the one or more processors are referred to herein collectively as “processor 702”). Processor 702 is operably connected to communication infrastructure 704 (e.g., a communications bus, cross-over bar, or network). The processor 702 may be embodied as any type of processor capable of performing the functions described herein. For example, the processor may be embodied as a single or multi-core processor(s), digital signal processor, microcontroller, or other processor or processing/controlling circuit.

The computing system 700 further includes a display interface 706 that forwards graphics, text, sounds, and other data from communication infrastructure 704 (or from a data buffer not shown) for display on display unit 708. The computing system further includes input devices 710. The input devices 710 may include one or more devices for interacting with the mobile device, such as a keypad, microphone, camera, as well as other input components, including motion sensors, and the like. In one embodiment, the display unit 708 may include a touch-sensitive display (also known as “touch screens” or “touchscreens”), in addition to, or as an alternative to, physical push-button keyboard or the like. The touch screen may generally display graphics and text, as well as provides a user interface (e.g., but not limited to graphical user interface (GUI)) through which a user may interact with the mobile device, such as accessing and interacting with applications executed on the device.

The computing system 700 further includes main memory 712, such as random access memory (RAM), and may also include secondary memory 714. The main memory 712 and secondary memory 714 may be embodied as any type of device or devices configured for short-term or long-term storage of data such as, for example, memory devices and circuits, memory cards, hard disk drives, solid-state drives, or other data storage devices. Similarly, the memory 712, 714 may be embodied as any type of volatile or non-volatile memory or data storage capable of performing the functions described herein.

In the illustrative embodiment, the mobile device may maintain one or more application programs, databases, media, and/or other information in the main and/or secondary memory 712, 714. The secondary memory 714 may include, for example, a hard disk drive 716 and/or removable storage drive 718, representing a floppy disk drive, a magnetic tape drive, an optical disk drive, etc. Removable storage drive 718 reads from and/or writes to removable storage unit 720 in any known manner. The removable storage unit 720 may represent a floppy disk, magnetic tape, optical disk, etc. which is read by and written to by removable storage drive 718. As will be appreciated, removable storage unit 720 includes a computer usable storage medium having stored therein computer software and/or data.

In alternative embodiments, the secondary memory 714 may include other similar devices for allowing computer programs or other instructions to be loaded into the computing system 700. Such devices may include, for example, a removable storage unit 724 and interface 722. Examples of such may include a program cartridge and cartridge interface (such as that found in video game devices), a removable memory chip (such as an erasable programmable read only memory (EPROM), or programmable read only memory (PROM)) and associated socket, and other removable storage units 724 and interfaces 722, which allow software and data to be transferred from removable storage unit 724 to the computing system 700.

The computing system 700 further includes one or more application programs 726 directly stored thereon. The application program(s) 726 may include any number of different software application programs, each configured to execute a specific task.

The computing system 700 further includes a communications interface 728. The communications interface 728 may be embodied as any communication circuit, device, or collection thereof, capable of enabling communications between the mobile device external devices (other mobile devices, a cloud-based service, an external computing system/server, etc.). The communications interface 728 may be configured to use any one or more communication technology and associated protocols, as described above, to effectuate such communication. For example, the communications interface 728 may be configured to communicate and exchange data with a server, an external computing system/server, and/or one other mobile device via a wireless transmission protocol including, but not limited to, BLUETOOTH™ communication, infrared communication, near field communication (NFC), radio-frequency identification (RFID) communication, cellular network communication, versions of IEEE 802.11 transmission protocol standards, and a combination thereof. Examples of communications interface 728 may include a modem, a network interface (such as an Ethernet card), a communications port, a Personal Computer Memory Card International Association (PCMCIA) slot and card, wireless communication circuitry, etc.

Computer programs (also referred to as computer control logic) may be stored in main memory 712 and/or secondary memory 714 or a local database on the mobile device. Computer programs may also be received via communications interface 728. Such computer programs, when executed, enable the computing system 700 to perform the features of the present invention, as discussed herein. In particular, the computer programs, including application programs 726, when executed, enable processor 702 to perform the features of the present invention. Accordingly, such computer programs represent controllers of computer system 700.

In one embodiment where the invention is implemented primarily in software, the software may be stored in a computer program product and loaded into the computing system 700 using removable storage drive 718, hard drive 716 or communications interface 728. The control logic (software), when executed by processor 702, causes processor 702 to perform the functions of the invention as described herein. In another embodiment, the invention may be implemented primarily in hardware using, for example, hardware components such as application specific integrated circuits (ASICs). Implementation of the hardware state machine so as to perform the functions described herein will be apparent to persons of ordinary skill in the relevant art(s). In yet another embodiment, the invention may be implemented using a combination of both hardware and software.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

1. A system for x-ray imaging comprising:
a source module comprising an x-ray source;
a high voltage power source for running the x-ray source for pulse-based operation;
a system of x-ray opaque leaves that can be moved independently to provide an aperture for x-rays produced by the x-ray source;
an optical camera system capable of accurately determining the orientation and distance of a device of know size with a known layout of markers;
a detector module separate from the source module and comprising an electronic x-ray detector that produces x-ray image data from x-rays produced by the x-ray source;
a computing system capable of processing the x-ray image data from the x-ray detector to produce images, the computing system providing a user interface (UI);
a computer screen capable of displaying the images produced by the portable computer system with high dynamic range; and
a communication connection between at least the source module and the detector module.

2. The system of claim 1, where at least one of:
there is no mechanical connection between the x-ray source and the electronic x-ray detector; or
the communication connection between the source and detector modules is wireless.

3. (canceled)

4. The system of claim 1, where the images are diagnostic images, optionally wherein at least one of:
the diagnostic images are of minor extremities such as the hands and feet;
the diagnostic images are of the torso or other major body parts;
the diagnostic images are of the head or other major extremities;
the diagnostic images are made in various poses optionally including weight bearing poses;
the diagnostic images have variable magnification based on the relative positions of the x-ray source and the x-ray detector; or
the diagnostic images are for veterinary purposes.

5-10. (canceled)

11. The system of claim 1, where at least one of:
the images are tomosynthesis based 3D images from a series of individual images and the corresponding position and orientation information;
the resultant image is displayed on the computer screen in a similar manner to a conventional digital camera;
multiple x-ray images are aligned and used in a tomosynthesis reconstruction by way of an array of radio-detectable fiducials that are mounted to the anatomy of the subject;
multiple x-ray images are aligned and used in a tomosynthesis reconstruction by way of an array of optical fiducials that are mounted to the anatomy of the subject and aligned using the same optical tracking system used to align the detector to the x-ray source;
multiple x-ray exposures are stitched together into one larger diagnostic image by use of the optical tracking system and known displacement of a grid x-ray detector;
the system allows multiple x-ray exposures where both the detector and source are moved for each exposure but located and stitched together by the use of the optical tracking system determining the position of the source and the detector with known markers and the use of a second set of visible markers that are stationary with respect to the patient between captures, but remain in the optical field of view;
the system creates an x-ray image with a predetermined source and detector distance and orientation through the use of user cues to orient and position the source module with respect to the detector module;
an optical image is taken concurrently with the diagnostic x-ray image and stored as a single record including the orientation and distance information;
the x-ray images are combined with optical views to create a tomosynthesis reconstruction with a texture mapped surface from the optical images;
the texture mapped tomosynthesis reconstruction is used to align future additional images and data sets with visual markers on the patient's anatomy; or
a diagnostic image equivalent to a linear scan image is created throughout the use of user cues, prescribed camera motion, and a continually adaptive slit configuration of the x-ray opaque leaves.

12. The system of claim 1, where the markers are on extendable or moveable arms to improve visibility, wherein the movement of the marker extensions are instrumented to provide accurate positioning information that is conveyed to the optical tracking system.

13. (canceled)

14. The system of claim 1, where the x-ray source and/or the x-ray detector are temporarily, semi-permanently, or permanently mounted on an existing structure or general use support structure.

15-20. (canceled)

21. The system of claim 1, where the x-ray opaque leaves are continually and near-instantaneously adjusted using the information from the optical tracking system and a known relationship to the focal spot of the x-ray source to ensure only x-rays that are aimed for the detector are permitted to emanate from the source.

22. The system of claim 1, wherein at least one of:
the system will only initiate the x-ray source if the x-ray opaque leaves are in the proper orientation;
initiation of the x-ray source is prevented if the optical tracking system is not able to obtain adequate tracking information; or
initiation of the x-ray source is prevented if the x-ray source is determined to be too close to the detector.

23-24. (canceled)

25. The system of claim 1, where a diagnostic image maybe be exchanged or uploaded to a medical record repository or another device for review. optionally wherein at least one of:
the image is uploaded via a wired network that connects to the source or the detector module;
the image is uploaded via a wireless network that is different than the network that connects to the source or the detector module; or
the image is uploaded via a wireless network that is the same as the network that connects to the source or the detector module.

26-31. (canceled)

32. The system of claim 1, that directly integrates diagnostic images into a central repository and can retrieve or restore medical records from such a repository, optionally wherein at least one of:
the system does this through a hardwired network link;
the system does this through a wireless network interface; or
the system does this through the same wired or wireless interface that link the source and detector units.

33-35. (canceled)

36. The system of claim 1, that can be combined with multiple source and detector units to align and combine x-ray exposures and optical images for additional information, optionally wherein at least one of:
the system uses multiple source or detector units to increase the field of view;
the system uses multiple source or detector units to aid in tomosynthesis; or
the system uses multiple source or detector units to locally increase magnification around an anatomical area of interest.

37-39. (canceled)

40. The system of claim 1, that uses the user interface (UI) to prompt the user to alter specific conditions to improve or augment imaging performance, optionally wherein at least one of:
the UI is configured to determine the relative position between the source and the detector modules and prompt the user to position the source module for orthogonal imaging;
the UI is configured to determine the relative position between the source and the detector modules and prompt the user to position the source module for additional projections to increase the effective field of view;
the UI is configured to determine the relative position between the source and the detector modules and prompt the user to position the source module for additional projections in aid of building a data set for tomosynthesis;
the UI is configured to determine the relative position between the source and the detector modules and prompt the user to position the source module for ideal magnification;
the UI is configured to determine the relative position between the source and the detector modules and display the effective magnification;
the UI is configured to determine the relative position between the source and the detector modules and prompt the user to position the source module to comply with predetermined safety or functional parameters;
the UI is configured to determine the relative position between the source and the detector modules and prompt the user to position the source module to align with a previous imaging exercise; or
the UI is configured to determine the relative position between the source and the detector modules and prompt the user to position the source module to align with a previous imaging exercise based on a combination of the source and detector position and optical landmarks, either anatomically based or artificial markers.

41-48. (canceled)

49. The system of claim 1, that uses the user interface (UI) to alter specific conditions to improve or augment imaging performance, alter doss or other change other beneficial conditions, optionally wherein at least one of:
the touchscreen is used to set the energy or beam current of the source;
markers manipulated on the screen are used to collimate the x-ray beam to an area smaller than the full detector size; or
markers manipulated on the screen to collimate the x-ray beam to an area smaller than the full detector size and indicates the effective area in real time on the screen, in some instances as an overlay on the optical image.

50-52. (canceled)

53. The system of claim 1, that incorporates additional sensing technologies for alignment and imaging, optionally wherein at least one of:
the system incorporates a lidar sensor;
the system incorporates at least one stereoscopic camera;
the system incorporates an infrared or thermal camera;
the system incorporates an ultrasonic distance measurement device;
additional sensor information is used to assist in a tomographic or tomosynthetic reconstruction; or
additional sensor information is used to assist in a tomographic or tomosynthetic reconstruction.

54-59. (canceled)

60. The system of claim 1, where at least one of:
the UI is separable from both the source and detector module;
the UI is either temporarily or permanently fixed to the source module; or
the UI is on another piece of hardware, such as a tablet or smartphone.

61-62. (canceled)

63. The system of claim 1, wherein at least one of:
the x-ray source comprises a carbon nanotube based x-ray source;
the x-ray source does not require external cooling;
the x-ray source can be rapidly turned on and off;
the system of x-ray opaque leaves is motor actuated;
the system of x-ray opaque leaves includes confirmatory position sensing;
the electronic x-ray detector comprises a solid-state flat panel detector, preferably wherein the electronic x-ray detector has a wide dynamic range, a small pixel size, and a large active area; or
the aperture includes a quadrilateral aperture.

64-68. (canceled)

69. The system of claim 1, wherein both the source module and the detector module are handheld modules, optionally wherein at least one of:
the power source is part of the source module;
the system of x-ray opaque leaves is part of the source module;
the optical camera system is part of the source module; or
the computer screen is part of the source module.

70-75. (canceled)

76. An x-ray source module for a portable x-ray system, the x-ray source module comprising:
a computing system;
an x-ray source;
a system of x-ray opaque leaves that can be moved independently to provide an aperture for x-rays produced by the x-ray source; and
a communication interface for communication with a separate x-ray detector.

77. An x-ray source module for a portable x-ray system, the x-ray source module comprising:
a housing configured to attach to a separate portable computer;
an x-ray source attached to the housing;
a system of x-ray opaque leaves that can be moved independently to provide an aperture for x-rays produced by the x-ray source; and
a communication interface for communication with the portable computer.

78. The x-ray source module of claim 77, wherein the x-ray source module is configured to receive at least some electrical power from the portable computer.
